(19) United States
(12) Patent Application Publication
(10) Pub. No.: US 2005/0055047 A1
(43) Pub. Date: Mar. 10, 2005

(54) Endovascular snare for capture and removal of arterial emboli

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 10/935,521

(22) Filed: Sep. 7, 2004

Related U.S. Application Data
(60) Provisional application No. 60/500,277, filed on Sep. 4, 2003.

(51) Int. Cl. A61M 29/00

(52) U.S. Cl. 606/200

(57) ABSTRACT

A snare for capture and removal of arterial emboli is disclosed. The snare is formed from a basket of interlaced filamentary members or a skeleton of interconnected flexible members. The basket has an opening at one end. A tongue extends from the basket adjacent to the opening, the tongue being offset from the basket centerline. A tether is attached to the tongue. The basket is collapsible into a collapsed configuration for delivery within an artery via a catheter. The basket is resiliently biased to expand into an open configuration upon release from the catheter. The tongue has a leading edge that engages the embolus and separates it from the artery when the snare is drawn toward it using the tether.

### FIELD OF THE INVENTION

This invention relates to devices for the treatment of strokes by capturing and removing the embolus or clot causing the stroke.

### BACKGROUND OF THE INVENTION

An ischemic stroke results when an artery carrying blood to a portion of the brain becomes blocked by an embolus. The embolus may be a blood clot or a fatty deposit which has broken free and is transported by the blood stream through the vascular system until it lodges in an artery within the brain that is too small to allow it to pass. The embolism or blockage of the artery reduces or totally halts the flow of blood to that portion of the brain normally fed by the now blocked artery, often with catastrophic consequences.

Each year, over 600,000 people in the United States suffer strokes and 27% of them die as a result. Only 10% of stroke victims achieve a full recovery, and 40% have moderate to severe impairments such as blindness, paralysis of the limbs, loss of speech function and loss of cognitive functions resulting from the death of oxygen-starved brain tissue.

It is preferred to take preventive measures against the occurrence of strokes. If detected early enough, a stroke may be treated with thrombolytic drugs which break up clots and operate to restore blood flow to the brain. Such treatment is not without increased risk of bleeding, however, which can cause additional brain damage. If the stroke victim arrives at a hospital too late for thrombolytic treatment (as most do), it is still advantageous to remove the blockage even though it will not restore the lost cerebral function or the dead tissue. Removal of the blockage will lessen the likelihood of additional strokes and prevent secondary effects, such as the release of excitotoxins by damaged neurons, cerebral edema as well as alterations in blood flow around the affected region, all of which contribute to additional neuronal death.

There is clearly a need for a minimally invasive device and technique for treating arterial embolisms by removing the emboli that cause strokes. Such a device will mitigate the risk of further strokes and further injury without itself presenting an increased risk of brain damage.

### SUMMARY OF THE INVENTION

The invention concerns an endovascular snare adapted for the capture and removal of emboli from a vascular vessel. The snare comprises a basket surrounding a central space and having an opening at one end. The opposite end is closed. The opening provides access to the central space for receiving the emboli. A tongue is attached to the basket adjacent to the opening and projects outwardly therefrom. The tongue has a leading edge for engaging and separating the emboli from the vessel. A tether is attached to the tongue for drawing the leading edge into engagement with the emboli, the tether being offset from a centerline of the basket.

Preferably, the basket is formed from a plurality of flexible, resilient interlaced filamentary members. The basket has a collapsed configuration facilitating percutaneous delivery into the vessel, and the basket is expandible into an open configuration for receiving the emboli. The filamentary members are resiliently biased so as to expand the basket into the open configuration in the absence of restraining forces holding the basket in the collapsed configuration.

In an alternate embodiment, the basket comprises a skeleton formed from a plurality of flexible, resilient members connected to one another end to end. The skeleton also has a collapsed configuration facilitating percutaneous delivery into the vessel and is expandible into an open configuration for receiving the emboli. The members are resiliently biased so as to expand the skeleton into the open configuration in the absence of restraining forces holding the skeleton in the collapsed configuration.

The basket may be decoupled from the tether. This is accomplished using a first eyelet positioned on the tongue and a second eyelet positioned on the basket at the opposite end. The eyelets are adapted to receive the tether through them. A blocking body is affixed to the tether. The blocking body is positioned between the first and second eyelets and engages the first eyelet upon drawing of the tether in a direction away from the basket. This allows the basket to be drawn in the same direction as the tether. The blocking body engages the second eyelet upon pushing of the tether in a direction toward the basket, which allows the basket to be pushed in the same direction as the tether.

The invention also includes a method of removing an embolus lodged within a vascular vessel using a snare deployed from a catheter. The method includes the steps of:
(A) inserting the catheter through the vessel and positioning the tip of the catheter past the embolus;
(B) deploying the snare from the catheter, the snare expanding into the open configuration;
(C) drawing the catheter away from the embolus and positioning the catheter tip on an opposite side of the embolus from the snare;
(D) drawing the snare toward the embolus;
(E) capturing the embolus within the basket; and
(F) removing the catheter and the snare from the vessel.

### BRIEF DESCRIPTION OF THE DRAWINGS

FIG. 1 is a perspective view of an endovascular snare according to the invention;

FIG. 2 is a side view of another embodiment of an endovascular snare;

FIG. 3 is a side view of yet another embodiment of an endovascular snare;

FIG. 4 is a side view of again another embodiment of an endovascular snare;

FIGS. 5-10 illustrate a method of removing emboli from a vessel;

FIGS. 11-13 illustrate another method of removing emboli from a vessel;

FIGS. 14 and 15 are further illustrations of a method of removing an embolus from an artery; and

FIGS. 16 and 17 are longitudinal sectional views of another embodiment of an endovascular snare.

### DETAILED DESCRIPTION OF THE PREFERRED EMBODIMENTS

FIG. 1 shows an endovascular snare 10 according to the invention. Snare 10 comprises a basket 12 having a sidewall 14 formed from a plurality of flexible, resilient filamentary members 16. The sidewall 14 surrounds and defines a central space 18 for receiving and holding arterial emboli causing a stroke. Basket 12 is preferably elongated and has an opening 20 at one end providing access to the central space 18, the other end 22 being closed. Elongated baskets are more stable when positioned within an artery or other vessel and the stability ensures that the opening 20 will remain aligned substantially coaxially with the artery to present the maximum area of the opening to receive the embolus.

Opening 20 is preferably flared to assume a funnel shape and has a tongue 21 projecting outwardly from the basket 12. The tongue 21 has a leading edge 23 adapted to engage an embolus and separate it from a vessel wall, the tongue directing the embolus into the flared opening 20 of basket 12 for capture and subsequent removal. By asymmetrically engaging the embolus with the tongue 21, the force necessary to separate and capture the embolus is reduced. The flared, funnel shape of opening 20 helps guide the embolus into the basket 12. Preferably, the tongue 21 is integrally formed with the sidewall 14 of the basket 12 from the same filamentary members 16 as the sidewall.

A tether 24 is attached to the tongue 21. Preferably, tether 24 is integrally formed with the tongue and the sidewall 14 and comprises extensions of the filamentary members 16. This provides a strong attachment of the tether 24 to the basket 12 and avoids the creation of stress concentrations or failure initiation points which would otherwise occur if a separate tether were attached to the basket, for example, using sutures, adhesive or fasteners. In the embodiment shown in FIG. 1, the tether 24 is an extension of the leading edge 23 and is offset from the centerline of the basket 12. An offset tether 24 keeps the opening 20 clear to permit capture of the embolus.

Filamentary members 16 are preferably interlaced by braiding to provide a snare having low bulk, high flexibility and resilient biasing. Braided structures have low bulk as compared with knitted or woven structures. Low bulk is advantageous because it permits the snare 10 to pass through smaller catheters than would be possible for a snare of the same size which was woven or knitted, allowing the device to be delivered by small catheters to relatively small arteries.

Flexibility of braided structures is afforded because the filamentary members are free to slide over one another at their cross over points, unlike woven or knitted structures wherein the filamentary members tend to constrain one another. Flexibility is desired so that the snare does not significantly increase the stiffness of the catheter and thereby inhibit its ability to traverse curved arteries and be guided into the branch where the embolus is lodged.

Resilient biasing of the snare ensures that it will expand from a collapsed configuration when released from the catheter and reliably assume an open configuration to receive the embolus within its central space 18 as described in detail below. Unlike woven structures wherein only the weft yarns contribute significantly to biasing which expands the device radially outwardly, all of the filamentary members comprising the braided snare are resiliently biased and effect expansion.

Braided tubular structures such as the basket 12 of the snare 10 also exhibit a characteristic known as the “trellis effect”, whereby the basket is caused to contract radially when a lengthwise tensile force is applied and expand radially when a lengthwise compressive force is applied. This characteristic allows the snare 10 to be conveniently configured into a collapsed configuration for packing into a catheter by applying a lengthwise tensile force to the snare, thereby reducing its diameter.

Filamentary members 16 may comprise a bio-compatible metal having a high yield strength to provide flexibility and resilience. Flexibility is advantageous so that the snare does not adversely stiffen the catheter and also so that it can bend to accommodate the shape of a vessel such as an artery in which it is deployed. Resilience allows the filamentary members 16 to be biased so that they cause the snare 10 to expand in the absence of restraining forces to assume the open configuration shown in FIG. 1. Biasing of metal filamentary members may be accomplished conveniently before or during the braiding process by cold working the filamentary members so they are biased into a desired curvature or by heat treatment applied to the filamentary members while they are braided over a mandrel. Candidate metals for the filamentary members 16 include stainless steel, nickel-titanium alloys such as nitinol, cobalt based alloys such as elgiloy, as well as titanium, all of which are compatible with living tissue and are readily biasable and flexible. Monofilaments are preferred because they provide greater biasing force than multi-stranded filaments, thus ensuring reliable expansion into the open configuration.

Polymers may also be used to form the filamentary members 16. The requirements for polymers are substantially the same as for metals in that the polymer material must be compatible with human tissue and produce a flexible and resilient filament. Polyester, polypropylene, nylon and polytetrafluoroethylene are all feasible materials for forming polymer filamentary members 16.

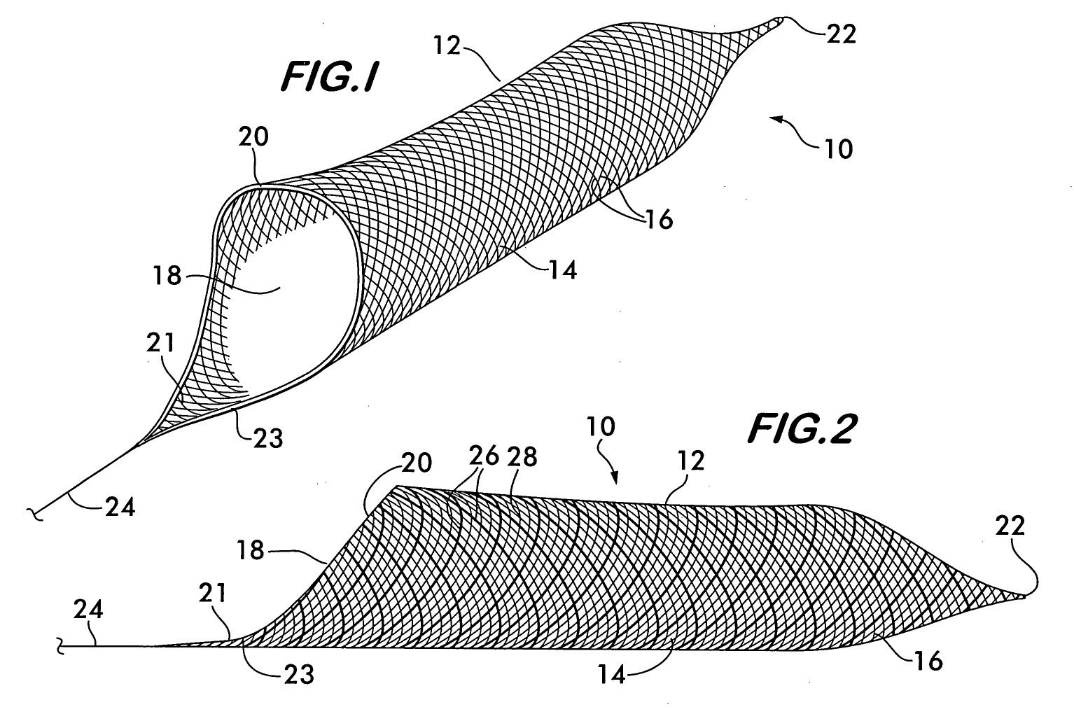

Polymer filamentary members may be used instead of or in conjunction with metal filamentary members to form the basket 12. In the example shown in FIG. 2, metal filamentary members 26 are inter-braided with polymer filamentary members 28 to augment the biasing forces of the polymer members and ensure expansion of the basket 12 into the open configuration. The presence of the polymer filamentary members 28 provides a less stiff basket 12 than would be obtained with an all metal design.

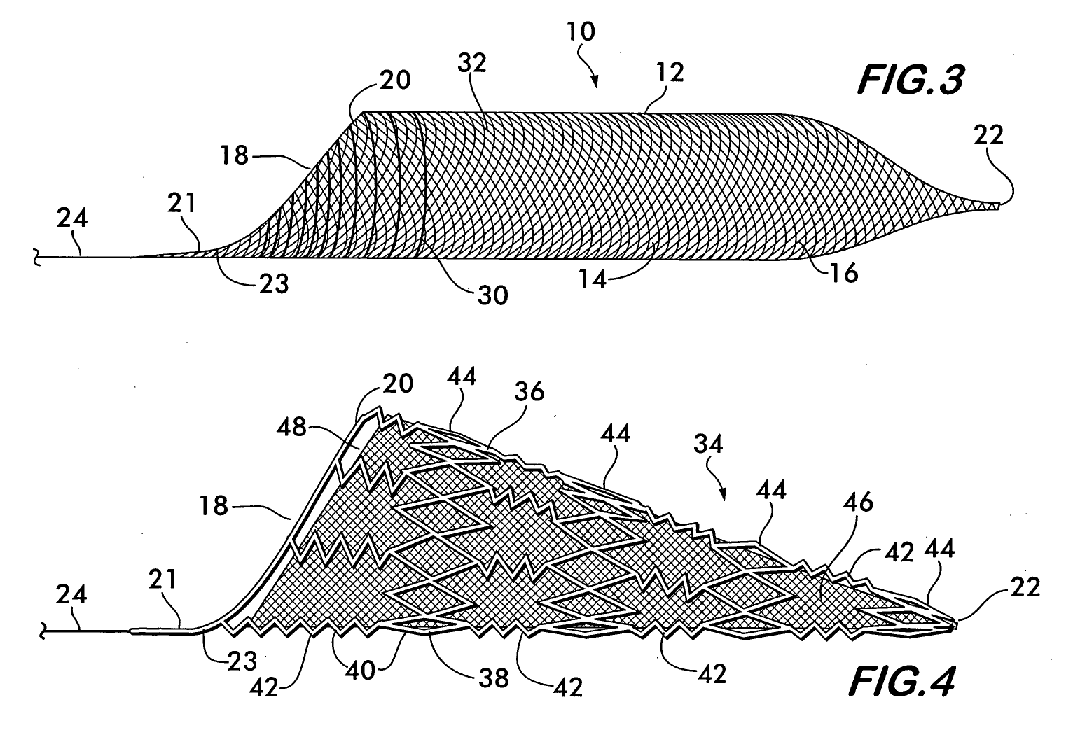

FIG. 3 shows another example of a basket 12 wherein the sidewall 14 surrounding the opening 20 is dominated by a relatively dense braid of metal filamentary members 30, the density of the braid of metal filamentary members 30 diminishing with distance from the opening 20 and having polymer filamentary members 32 predominating. The relatively dense braid of metal filamentary members 30 increases the biasing force at the opening 20 to ensure that it expands fully into the open configuration, and greater flexibility is afforded by the polymer filamentary members 32 forming the greater part of the length of basket 12. It is also feasible to augment the biasing force at the opening 20 by increasing the area moment of inertia of the filamentary members 16 in the region of the opening 20. This may be accomplished by, for example, increasing the gage of the filaments uses to form the filamentary members. Metal filamentary members also provide greater radiopacity, allowing the snare to be observed using fluoroscopic techniques.

FIG. 4 shows another snare embodiment 34 according to the invention. Snare 34 comprises a basket 36 that is laser cut from a funnel shaped tube originally having a substantially continuous sidewall. Manufacturing techniques using precise laser cutters controlled by computer enable the majority of the material from the funnel to be removed leaving only a skeleton 38 of interconnected members 40 forming basket 36. Skeleton 38 is preferably comprised of metal such as nitinol or elgiloy, which are flexible and resilient, to facilitate collapsing of the snare for delivery by a catheter and expansion of the snare into the open configuration shown in FIG. 4 when positioned within a vessel. Other bio-compatible metals such as stainless steel or titanium are also feasible, as are polymers such as polyester, polypropylene and nylon. The basket 36 of snare 34 surrounds a central space 18 and has an opening 20 at one end providing access to the central space, the opposite end 22 being substantially closed. A tether 24, again offset from the centerline of the snare, is attached to a tongue 21 positioned adjacent to opening 20, the tongue having a leading edge 23.

An advantage to cutting the skeleton 38 from a tube or funnel is that is allows more precise control of the resilient biasing forces that govern radial and lengthwise expansion and compression of the snare 34 when it is compressed to fit within and traverse a catheter and when it expands within a vessel to capture an embolus. Control of the biasing forces is afforded by cutting the funnel so that there are skeletal regions 42 having greater flexibility in the axial direction lengthwise along the snare and skeletal regions 44 having greater radial flexibility in the circumferential direction around the snare. Axially flexible regions 42 are created by orienting interconnected members 40 so that bending stresses predominate when the member is compressed or stretched along the length of the snare 34, as illustrated by the lengthwise zig-zag pattern of portions 42. Likewise, radially flexible regions are created by interconnecting members 40 in a diamond pattern seen in regions 44 which extend circumferentially around the snare. Axially flexible regions 42 will deform like an accordion lengthwise along the snare 34 to allow the snare to stretch and compress lengthwise, while radially flexible regions 44 deform, again like an accordion, but about the circumference of the snare. The parameters controlling the biasing forces of the regions 42 and 44 are the material comprising the members 40, the area moment of inertia of members 40, and the number of members and length of each member. Material having a high elastic modulus will have greater biasing force, as will members having greater area moments of inertia.

It may be advantageous to combine the snare 34 with a sack 46. Sack 46 may be woven, knitted or braided from polymer filamentary members such as polyester, polytetrafluoroethylene, polypropylene or nylon. Alternately, the sack may be a substantially continuous membrane made, for example, from expanded polytetrafluoroethylene. Sack 46 has an opening 48 aligned with the opening 20 of the snare 34. The sack 46 preferably is elongated and may form a liner located within basket 36 of snare 34, or may constitute a cover surrounding the outside of the basket. Sack 46 works to ensure that the embolus is captured and contained within the snare 34 in the event that the skeleton 38 is too porous to reliably contain the embolus.

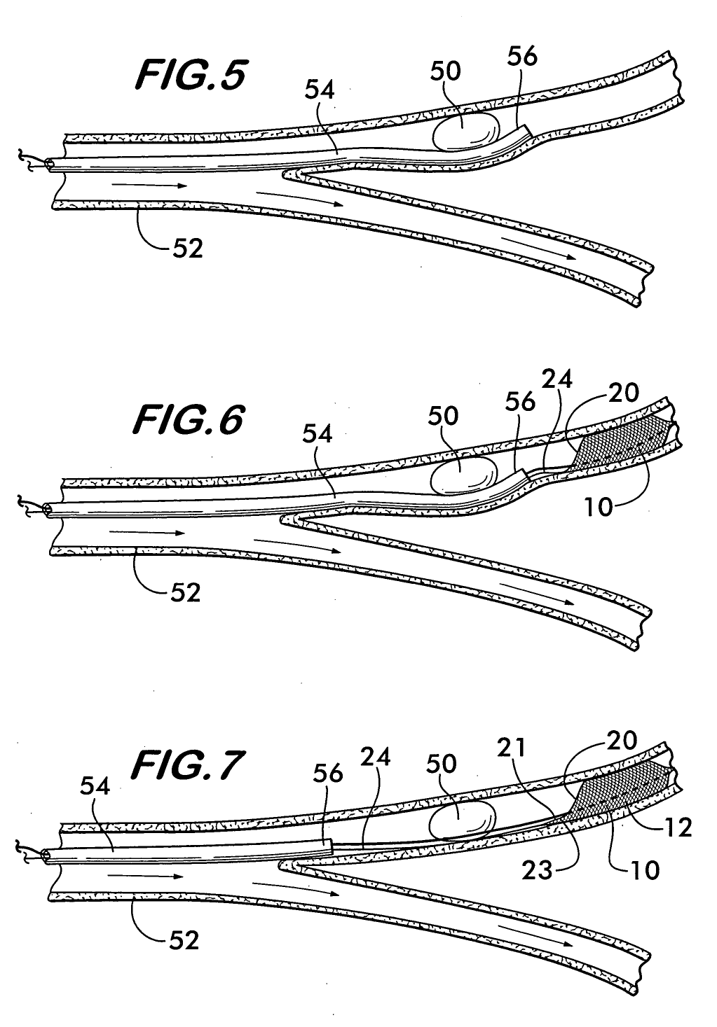

FIGS. 5-10 illustrate one technique for removing an embolus from an artery using a snare 10 according to the invention. As shown in FIG. 5, an embolus in the form of a blood clot 50 is lodged in an artery 52. A catheter 54 is inserted within the artery 52, its tip 56 being pushed past the clot 50. As shown in FIG. 6, the snare 10 is pushed out from the catheter 54 into the artery 52 whereupon it resiliently expands into an open configuration with its opening 20 facing the clot 50 and its tether 24 leading back into the catheter 54. The snare and its tether are capable of moving independently of the catheter 54 under the control of the surgeon performing the procedure.

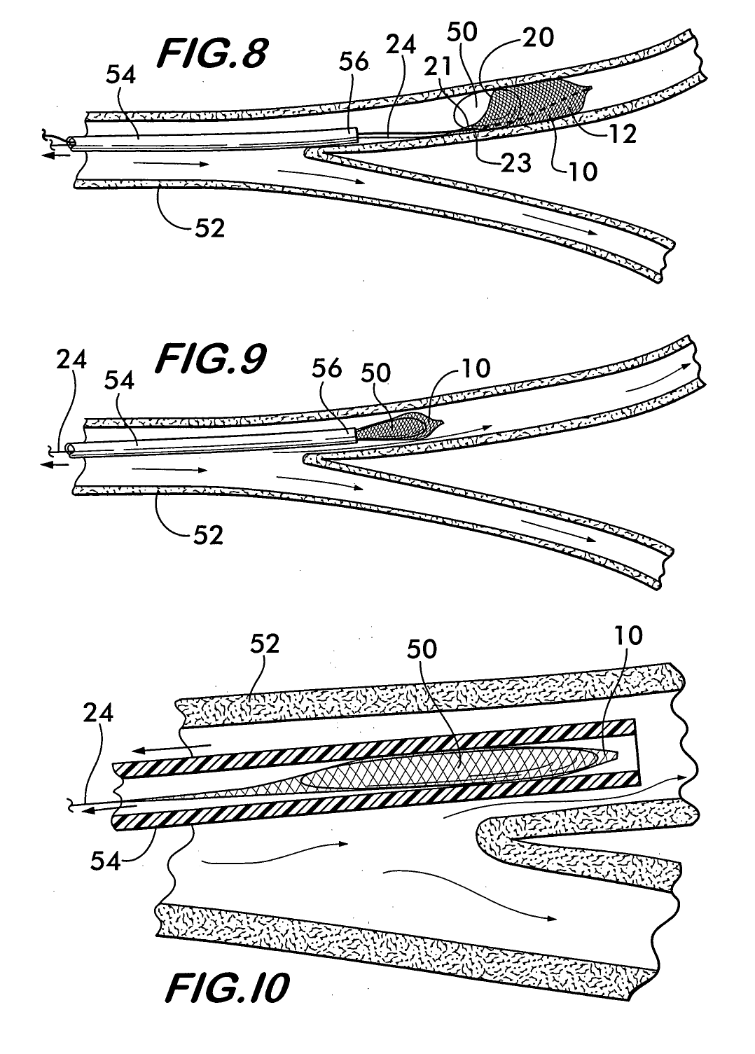

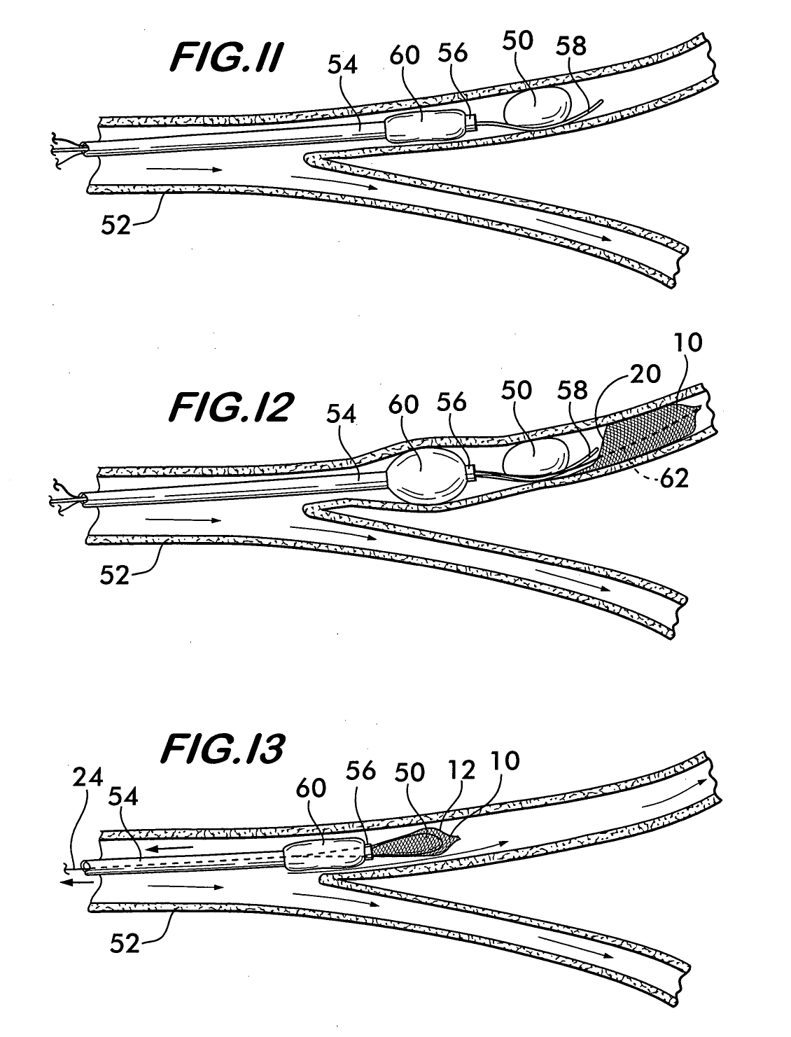

As further shown in FIG. 7, the catheter 54 is partially withdrawn to position its tip 56 on an opposite side of the clot 50 from the snare 10. Next, as shown in FIG. 8, the snare 10 is drawn toward the catheter using tether 24. The clot 50 is separated from the artery 52 by the action of tongue 21 with leading edged 23 and enters the opening 20 to be captured within the elongated basket 12. As shown in FIG. 9, the snare and clot are then withdrawn from the artery 52 along with the catheter. Upon withdrawal, the snare 10 may extend partially out of catheter 54 as depicted in FIG. 9, or as shown in FIG. 10, the snare and the clot 50 may be drawn into the catheter. The snare 10 again assumes its collapsed configuration as it enters the catheter. Note that the tapered shape preferred for the tongue 21 operates in conjunction with the opening 20 to collapse the opening, thereby also initiating collapse of the snare 10 so that it may be smoothly drawn back into the catheter for removal from the artery 52.

FIGS. 11-13 show another method of removing an embolus blocking a vessel. Again, as shown in FIG. 11, a blood clot 50 is lodged in an artery 52. A catheter 54 having a tip 56 is guided over a guide wire 58 to position tip 56 proximate to clot 50. Catheter 54 has a balloon 60 positioned near its tip 56. As shown in FIG. 12, the balloon 60 is inflated to expand the artery 52 and allow the snare 10 to be pushed past the clot 50 by a pusher element 62 within the catheter 54. Once past the clot 50, the snare 10 expands with its opening 20 facing the clot. As shown in FIG. 13, tether 24 is then used to draw the snare 10 toward the catheter, the opening 20 receiving clot 50 which is then captured in basket 12. Expansion of balloon 60 may also result in a back flow of blood in artery 54, the back flow facilitating movement of the clot and snare towards the catheter tip 56. Upon capture of the clot within basket 12, the balloon 60 is deflated and catheter 54 and snare 10 containing clot 50 are then withdrawn from the artery 52.

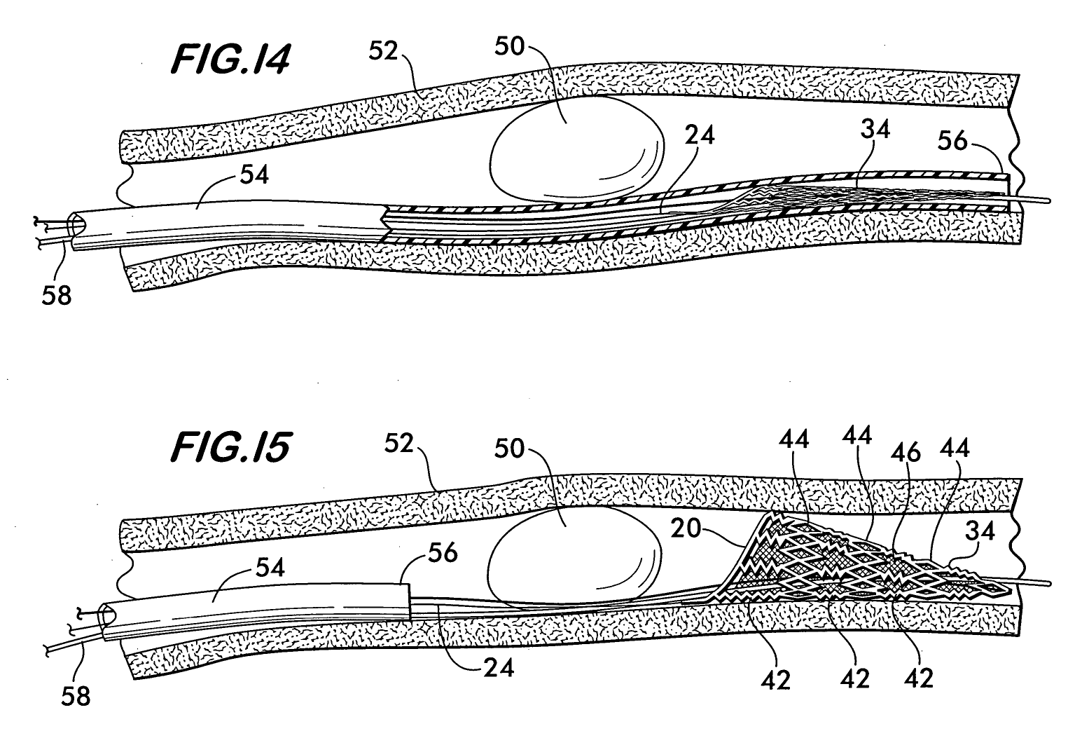

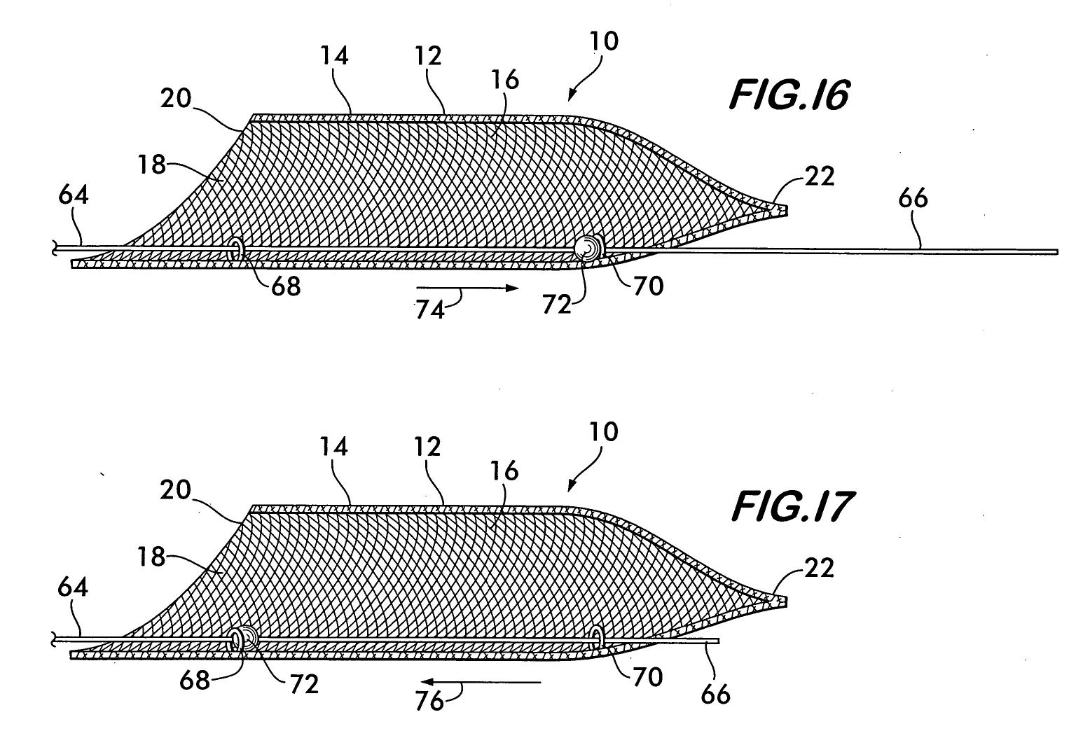

FIGS. 14 and 15 illustrate use of the snare embodiment 34 to remove a clot 50 from an artery 52. As shown in FIG. 14, snare 34 is delivered in a collapsed configuration within catheter 54 guided along a guide wire 58 positioned within the artery 52. The tip 56 of catheter 54 is positioned past the clot 50 and the snare 34 is pushed out of the catheter.

As shown in FIG. 15, the snare 34 expands to an open configuration once free of catheter 54. Resilient biasing forces in axially flexible regions 42 expand the snare lengthwise and resilient biasing forces in the radially flexible regions 44 expand the snare circumferentially to form the opening 20 which faces the clot 50. Tether 24 is then used to draw the snare 34 toward the clot 50 and capture it within sack 46 for eventual removal along with the catheter 54.

FIGS. 16 and 17 show a snare 10 wherein the tether 64 is decoupled from the basket 12, i.e., the tether may move independently of the basket over a limited distance along its length. The decoupled tether 64 preferably extends through the central space 18 and has excess length 66 which projects outwardly from closed end 22. Motion of tether 64 is constrained by eyelets 68 and 70 that are positioned on tongue 21 and sidewall 14 near closed end 22 and extend into central space 18. The eyelets 68 and 70, shown in the form of loops extending from the tongue 21 and the sidewall 14, cooperate with a blocking body 72 affixed to the tether 64 between the guide stops and act to limit the motion of the tether 64 relative to basket 12. The blocking body 72 preferably has a spherical shape with a diameter greater than that of the eyelets 68 and 70 to ensure positive engagement.

Cooperation between the blocking body 72 and eyelets 68 and 70 permit the tether 64 to both push and pull the snare 10, allowing it to be readily maneuvered through catheters and past obstructions into a desired position. As shown in FIG. 16, when tether 64 is pushed toward snare 10 in the direction of arrow 74, the blocking body 72 traverses the length of basket 12 and engages eyelet 70, which acts as a stop to prevent further relative motion between the tether 64 and basket 12. Further pushing on tether 64 pushes the basket, allowing it to be, for example, pushed out of a catheter or past a clot in an artery. With the blocking body 72 engaged with eyelet 70, pushing on tether 64 takes advantage of the trellis effect associated with braided structures whereby the basket 12, being subjected to a force at its closed end 22, tends to extend lengthwise and collapse radially, allowing it to pass through narrow openings and other confined spaces.

Once in a desired position, for example, adjacent to a clot in an artery, the tether 64 is drawn in the opposite direction as indicated by arrow 76 in FIG. 17. Blocking body 72 traverses the length of basket 12 and engages eyelet 68, and the basket 12 is pulled toward the clot or other obstruction to be ensnared. Pulling the basket 12 from its open end causes it to assume an open configuration in compliance with its biasing and the clot may enter the central space 18 through opening 20.

Snares according to the invention provide a minimally invasive method for treating strokes by removing the embolism with minimum additional risk to the patient, thus, realizing the advantages of reduced potential for secondary effects such as further strokes, cerebral edema and the release of excitotoxins which would otherwise cause further brain damage and impairment.

1. An endovascular snare adapted for the capture and removal of emboli from a vascular vessel, said snare comprising:
a basket surrounding a central space and having an opening at one end, the opposite end being closed, said opening providing access to said central space for receiving said emboli;
a tongue attached to said basket adjacent to said opening and projecting outwardly therefrom, said tongue having a leading edge for engaging and separating said emboli from said vessel; and
a tether attached to said tongue for drawing said leading edge into engagement with said emboli, said tether being offset from a centerline of said basket.

2. An endovascular snare according to claim 1, wherein said tongue and said tether are integrally formed with said basket.

3. An endovascular snare according to claim 1, wherein said basket is formed from a plurality of flexible, resilient interlaced filamentary members, said basket having a collapsed configuration facilitating percutaneous delivery into said vessel, said basket being expandible into an open configuration for receiving said emboli, said filamentary members being resiliently biased so as to expand said basket into said open configuration in the absence of restraining forces holding said basket in said collapsed configuration.

4. An endovascular snare according to claim 3, wherein said filamentary members are interlaced by braiding.

5. An endovascular snare according to claim 4, wherein said filamentary members comprise monofilaments braided at a first density over a region surrounding said opening, said monofilaments being braided over a remainder of said basket at a second density less than said first density, said monofilaments surrounding said opening facilitating biasing of said basket into said open configuration at said opening.

6. An endovascular snare according to claim 5, wherein said monofilaments comprise metal.

7. An endovascular snare according to claim 1, wherein said basket comprises a skeleton formed from a plurality of flexible, resilient members connected to one another end to end, said skeleton having a collapsed configuration facilitating percutaneous delivery into said vessel, said skeleton being expandible into an open configuration for receiving said emboli, said members being resiliently biased so as to expand said skeleton into said open configuration in the absence of restraining forces holding said skeleton in said collapsed configuration.

8. An endovascular snare according to claim 7, wherein said basket has a funnel shape.

9. An endovascular snare according to claim 7, further comprising a sack attached to said skeleton, said sack having a sack opening aligned with said opening of said basket for receiving said emboli within said sack.

10. An endovascular snare according to claim 9, wherein said sack is positioned within said central space of said basket.

11. An endovascular snare according to claim 9, wherein said sack comprises a plurality of interlaced filamentary members.

12. An endovascular snare according to claim 1, further comprising:
a first eyelet positioned on said tongue;
a second eyelet positioned on said basket at said opposite end, said eyelets being adapted to receive said tether therethrough; and
a blocking body affixed to said tether and positioned between said first and second eyelets, said blocking body engaging said first eyelet upon drawing of said tether in a direction away from said basket and thereby allowing said basket to be drawn in the same direction as said tether, said blocking body engaging said second eyelet upon pushing of said tether in a direction toward said basket thereby allowing said basket to be pushed in the same direction as said tether.

13. An endovascular snare adapted for the capture and removal of emboli from a vascular vessel, said snare being movable through a catheter for positioning within said vessel, said snare comprising:
a basket surrounding a central space and having an opening at one end, the opposite end being closed, said opening providing access to said central space for receiving said emboli;
a tongue attached to said basket adjacent to said opening and projecting outwardly therefrom, said tongue having a leading edge for engaging and separating said emboli from said vessel, said tongue and said basket being formed from a plurality of flexible, resilient monofilaments braided together, said basket being collapsible into a collapsed configuration for passage of said basket through said catheter and into said vessel, said monofilaments being resiliently biased to expand said basket into an open configuration for receiving said emboli upon release thereof from said catheter; and
a tether attached to said tongue for drawing said leading edge into engagement with said emboli, said tether being offset from a centerline of said basket.

14. An endovascular snare according to claim 13, wherein a portion of said monofilaments comprise metal.

15. An endovascular snare according to claim 14, wherein a portion of said monofilaments comprise a polymer material.

16. A method of removing an embolus lodged within a vascular vessel using a snare deployed from a catheter, said method including the steps of:
inserting said catheter through said vessel and positioning the tip of said catheter past said embolus;
deploying said snare from said catheter, said snare expanding into said open configuration;
drawing said catheter away from said embolus and positioning said catheter tip on an opposite side of said embolus from said snare;
drawing said snare toward said embolus;
capturing said embolus within said basket; and
removing said catheter and said snare from said vessel.

17. A method according to claim 16, further comprising the step of drawing said snare with said embolus into said catheter.

18. A method of removing an embolus lodged within a vascular vessel using a snare deployed from a catheter having a balloon positioned near the catheter tip, said method including the steps of:
inserting said catheter through said vessel and positioning the tip of said catheter proximate to said embolus;
inflating said balloon to enlarge said vessel and block flow therethrough;
deploying said snare from said catheter to a position on an opposite side of said embolus from said catheter tip, said snare expanding into said open configuration;
drawing said snare toward said embolus;
capturing said embolus within said basket;
deflating said balloon; and
removing said catheter and said snare from said vessel.

19. A method according to claim 17, further comprising the step of drawing said snare with said embolus into said catheter.
